SEQUENCE LISTING (3 sequences)

SEQ ID NO: 1 - DNA, 21 bp, artificial sequence
  Nucleotide sequence of HCV viralprimer, 2CH primer
aactactgtcttcacgcagaa

SEQ ID NO: 2 - DNA, 21 bp, artificial sequence
  Nucleotide sequence of HCV viralprimer, 1TS primer
gcgacccaacactactcggct

SEQ ID NO: 3 - DNA, 19 bp, artificial sequence
  Nucleotide sequence of HCV viralprobe, TM416 probe
aacccgctcaatgcctgga

(19) United States
(12) Patent Application Publication
(10) Pub. No.: US 2010/0144014 A1
(43) Pub. Date: Jun. 10, 2010

(54) METHOD FOR AMPLIFYING HCV IN AEDES MOSQUITOES

(76) Inventors: Emmanuel Drouet, Corenc (FR); Yassine Rechoum, Grenoble (FR); Florence Fouque, Le Rove (FR)

Correspondence Address: THE NATH LAW GROUP, 112 South West Street, Alexandria, VA, 22314, US

(21) Appl. No.: 12/448,420

(22) PCT Filed: Dec. 20, 2007
(86) PCT No.: PCT/FR2007/052592

§ 371 Date: Feb. 4, 2010

(30) Foreign Application Priority Data
Dec. 22, 2006 (FR) 0655935

(51) Int. Cl. C12N 7/01; C12N 5/07

(52) U.S. Cl. 435/235.1; 435/348

(57) ABSTRACT

A process for amplifying hepatitis C virus (HCV) in vivo is provided. The method includes ingesting

a viral source by female Aedes vexans or Aedes caspius mosquitoes, and

rearing the mosquitoes for a time necessary to amplify the virus.

### TECHNICAL FIELD

This invention concerns the identification of new systems for amplifying the hepatitis C virus (HCV). It has been shown that certain mosquitoes of the Aedes genus allow effective replication of HCV in vivo. This provides promising prospects, particularly for vaccinology and the development of drugs against this infection.

### PRIOR ART

The hepatitis C virus (HCV) has been identified as being responsible for non-A non-B hepatitis which can develop into malignant chronic diseases, e.g. cirrhosis of the liver or hepatocellular carcinoma.

HCV is a member of the Hepacivirus genus of the Flaviviridae family, which are enveloped single strand RNA viruses and include those responsible for major epidemic diseases such as yellow fever (YF), dengue fever (DEN) and dengue haemorrhagic fever (DHF), Japanese encephalitis (JE), St. Louis encephalitis (SLE), West Nile fever (WN) and hepatitis C (HC), to mention only the most important.

Flaviviruses are transmitted by insect vectors in very different epidemiological ways. Some diseases are typically human (or linked to primates) and never affect animals e.g. DEN and DHF, while other infections are rather zoonotic and affect humans more or less accidentally e.g. JE, SLE and WN. Finally, certain Flaviviruses can circulate in epidemic form both in human and animal populations (YF). These different epidemiological methods of transmission nevertheless have common factors such as viral amplification in insect cells.

As far as HCV is concerned, the transmission routes nowadays well established are via blood products, drug addiction, sexual transmission and the nosocomial context. Nevertheless, the origin of 30 to 40% of HCV infections recorded in humans today is unexplained, in as far as none of the conditions listed above apply. Moreover, a high prevalence of HCV infection has been observed in hot, humid regions, such as Egypt and in southern Japan, near JEV. At the present time, no explanation has been given for this geographical distribution of the disease.

As far as anti-HCV vaccination strategies are concerned, they are currently based on two processes usual in the field: either Vaccinia or AAV (adeno-associated virus) recombinants, or anti-HCV vaccinotherapy strategies developed based on polypeptide derivatives of HCV proteins.

In the battle against type C viral hepatitis, the absence of a system allowing a high level of multiplication of the virus in vitro is a major obstacle hindering the development of a treatment and an effective vaccine. This may be explained by poor adaptation of the virus in Man, making it incapable of replicating in vitro in conventional cell models, such as the hepatocarcinoma line or primary hepatic cells.

Molecular constructions have been proposed to overcome this problem, particularly the replicon model, but they all share the defect of being alternative forms quite distant from natural C particles.

More exhaustively, the models currently available for amplifying HCV are as follows:

In vivo models of infection and viral replication are given in the review by Bartenschlager et al. (1). As far as conventional animal models are concerned, the only animal susceptible to HCV infection is the chimpanzee (P. troglodytes), in which the blood virus titre is high. Other primates (Tupaia belangeri chinensis) can be infected, but blood virus titres are low and intermittent. Another animal model available is the beige/nude/immunodeficient mouse with xenotransplantation of human hepatocytes (mouse trimera®) which has a high blood virus titre, but its success is linked to the lifespan of the transgene.

The review by Bartenschlager et al. (1) also reports the existence of cell culture systems. No cell line allowing a complete permissive cycle and the production of viral particles after conventional infection has been described (8). The only effective system allowing replication is that described by Bartenschlager and Lohmann (2): it consists of a subgenomic replicon system in which HCV genomic RNA is replicated and non-structural proteins (in particular protease-helicase and RNA polymerase) are expressed after transfection of Huh-7 cells by the cloned replicon. This system is used in antiviral screening. A considerable improvement was provided by Wakita et al. (11) whose replicon system (HCV-JFH1 clone) can produce complete infectious viral particles (after transfection into Huh-7 cells). Nevertheless, the system is not suitable at present for the production of a large biomass.

Finally, heterologous expression systems have been developed. These are firstly “Virus Like Particles” (VLPs), obtained from a baculovirus expressing the E1E2 heterodimeric envelope glycoproteins (4). Recently, the baculovirus system has been used on the basis of a recombinant containing a core protein cDNA+E1E2 gp, in an experiment immunising chimpanzees with a vaccine objective. Secondly, rhabdoviral (VSV, 6) or retroviral (HIV or MLV, 3) pseudotypes or pseudoparticles (pp) have been obtained. These fundamentally important systems are used to study viral entry via the E1E2 gp. They can only be used to study anti-HCV envelope neutralizing antibodies and are therefore of no interest in vaccinology.

It thus seems that none of the systems described is completely satisfactory and that there is still a need to develop new models which effectively amplify HCV.

DESCRIPTION OF THE INVENTION

To meet this need the invention thus concerns an in vivo process for amplifying the hepatitis C virus (HCV) consisting of the following stages:

a/ingestion of the viral source by female mosquitoes of the Aedes genus;

b/rearing the mosquitoes for the time necessary to amplify the virus.

The first step thus consists of getting the virus into the mosquito in the most effective way possible, which is by ingestion. After the mosquito has ingested the virus it arrives in the stomach, where it crosses the stomach wall and disperses in the haemocoel to reach the various organs, such as the ovaries and salivary glands, where it can multiply. As a consequence, the virus can be transmitted to future generations of mosquitoes or to humans when they are bitten.

Mosquitoes of the Aedes genus belong to the order Diptera, suborder Nematocera, family Culicidae, subfamily Culicidae, tribe Aedini. Females have characteristic short palps and non-feathery antennae.

For this invention, the species Aedes vexans and Aedes caspius are used.

To advantage, the wild strains of these species are used for the invention. The population used in the process is the result of breeding from material (eggs, larvae or adults) taken directly from the field, in the biotope favoured by these two species. To advantage the individuals used belong to generations F0, F1 or F2.

As already stated, contact between the viral source and the mosquitoes is by ingestion. To advantage, the viral source is prepared with red blood cells (e.g. from sheep), possibly supplemented with ATP, to increase the attractiveness for female mosquitoes.

In practice, ingestion occurs therefore during a blood meal taken by the female mosquitoes.

To advantage, ingestion is carried out using the engorgement technique. This particularly effective technique is well known to entomologists and is described for example by Fouque et al. (7). In principle, a blood meal is provided for the female mosquitoes in an ‘engorger’ which maintains the meal at 37° C. The engorger is covered by a skin through which the mosquito inserts its proboscis and takes the meal.

In an advantageous embodiment of this technique, chicken skin is used.

According to the invention, the viral source includes a “native” virus (virulent), or to advantage an attenuated virus. With a virulent virus infection is reproduced, so that it therefore provides a first choice of experimental model. In a more applied way, an attenuated virus is of particular interest in vaccine or antibody production.

According to a preferred embodiment of the invention, the viral load comes from a serum, to advantage of human origin, e.g. that of a subject with chronic hepatitis caused by HCV genotype 1b. The classic titre of such a serum is generally around 2^{E}6 copies/ml.

For the invention, it was determined that the contact must occur with at least 1000 copies of the virus per mosquito, to advantage 1500 copies of the virus per mosquito.

The second step of the process according to the invention consists of rearing the mosquitoes for a given period. They are reared under controlled conditions, particularly as concerns temperature and humidity. The duration of the rearing period is determined according to the following criteria:
It must be less than the average lifespan of the species of mosquito in captivity
It must allow at least one cycle of extrinsic amplification to occur in the mosquito, and preferably permit a maximum rate of virus replication.

For the invention, amplification appears to be optimal with rearing for between 15 and 30 days for the Aedes tested, with an advantageous mean value for a rearing period of about 20 days.

Recovering the virus may require it to be extracted, particularly in the form of viral particles, from the tissues of the mosquitoes. The tissues preferred are the ovaries and the salivary glands.

The invention thus provides a process for amplifying HCV, occurring in vivo in specific species of mosquito, giving completely unexpected and remarkable results.

It was indeed known from FR 2793 258 and Germi et al. (9) that HCV could be produced in vitro in insect cells, though with inadequate results.

Moreover, previous experiments carried out in vivo on mosquitoes belonging to the species Aedes aegypti, Aedes albopictus, Anopheles stephensi and Culex quinquefasciatus proved negative (10, 5), the results being consistent with the concept of long coadaptation and great specificity between virus and mosquito vector. They highlight the merits of the present invention.

As already stated, the process according to the invention has promising prospects, including for large-scale production of HCV in vitro.

The process according to the invention thus gives access to tissues, particularly from the ovary or salivary gland, or to Aedes cells, to advantage from Ae. caspius or vexans, HCV carriers.

These cells can be used as they are for the large-scale production of HCV in vitro.

In another embodiment, the invention also concerns a process for obtaining cells producing HCV in vitro. Aedes cells carrying HCV, obtained using the process according to the invention, are cocultivated with AP61 or C6/36 type cells. AP61 and C6/36 type cells have been described by Germi et al. (9). They are continuous, well-defined, mosquito cell lines, in particular from Aedes pseudoscutellaris. The purpose of coculturing is to transfer the HCV into these cell lines. Thus the invention also concerns mosquito cells producing HCV in vitro obtained at the end of this process.

These two cell sources of HCV offer considerable prospects, in particular of producing a new kind of viral particle (coming from Aedes) for the production of specific antibodies or the preparation of prophylactic or therapeutic vaccines.

### EXAMPLES OF EMBODIMENTS

The invention and the advantages resulting from it are better illustrated by the following examples of embodiments and the attached figures. However, theses examples are in no case limiting.

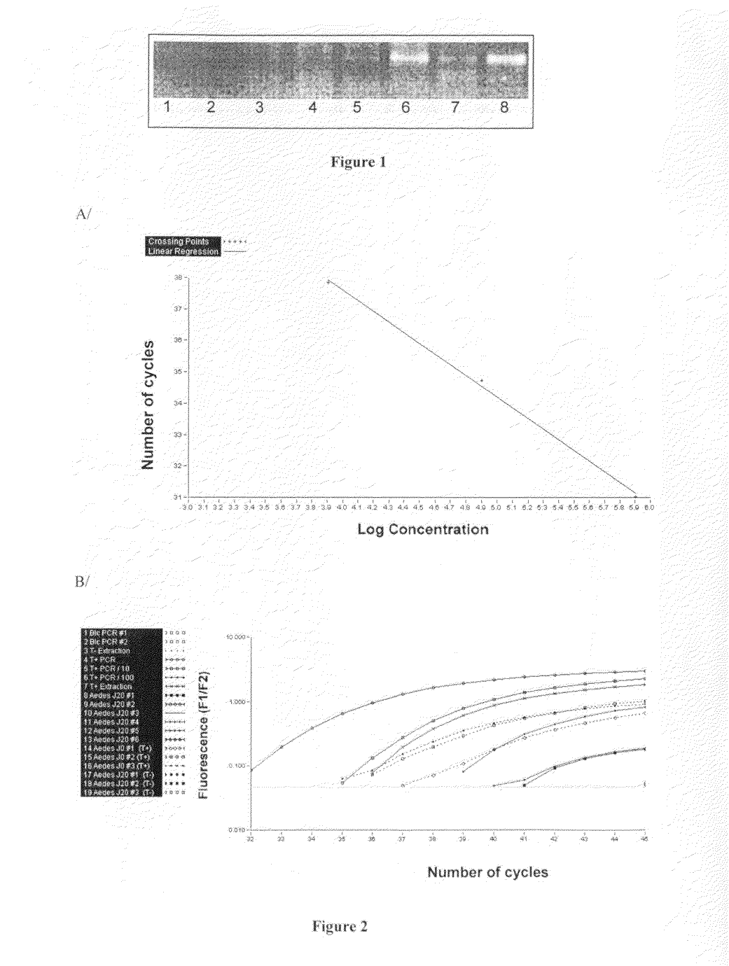

FIG. 1 shows a gel illustrating the detection of the viral RNA in HCV infected Aedes vexans mosquitoes, after extraction of total RNAs and classic RT-PCR with the DNA being stained with ethidium bromide. Band 1). PCR negative control; band 2) Mosquito negative control; bands 3, 4) Day 0; band 5) Day 14; bands 6, 7, 8) Day 21.

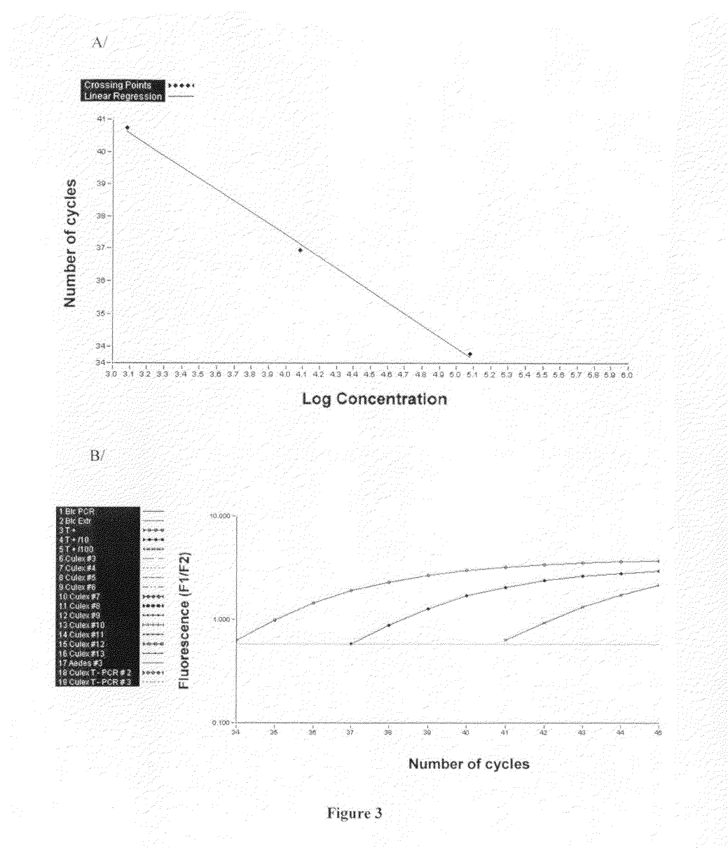

FIG. 2 illustrates detection of the viral RNA by qRT-PCR after extraction of total RNAs from Aedes (vexans+caspius) mosquitoes after rearing for 24 days. The standard graph was produced from a logarithmic dilution of a viral RNA extract of known titre (120,000 copies/run to 1,200 copies/run) (FIG. 2A). In FIG. 2B, the positive controls are identified by (J0, T+), and the individuals likely to have replicated the HCV after 24 days by (J20). Also given for each sample are (i) the number of cycles after which the viral RNA was detected and (ii) its initial concentration relative to the standard graph. The graph in FIG. 2B shows how detection of the signal changes for each sample.

FIG. 3 illustrates detection of the viral RNA by qRT-PCR after extraction of total RNAs from Culex (pipiens) mosquitoes after rearing for 28 days. The standard graph was produced from a logarithmic dilution of a viral RNA extract of known titre (800,000 copies/run to 8,000 copies/run) (FIG. 3A). Also given for each sample are (i) the number of cycles after which the viral RNA was detected and (ii) its initial concentration relative to the standard graph. The graph in FIG. 3B shows how detection of the signal changes for each sample.

### EXPERIMENTAL PART

### I—Experimental Infections

### 1. Rearing the Species Tested

The individuals tested came from insects reared from mosquitoes (eggs, larvae and adults) collected in the field in the urban area of Marseilles for the Culex pipiens species and in the Tour du Valat reserve in the Camargue for the Aedes vexans and Aedes caspius species. The insects were reared in the ENSAM (Ecole Nationale Supérieure d'Arts et Métiers) premises at the Domaine du Merle, in Salon de Provence. Rearing is in a classic insectarium, meeting rearing standards. The larvae are fed brewers yeasts and fish food and the adults are fed a sucrose solution. The Culex and Aedes blood meals are composed of washed sheep red cells, washing being in order to eliminate interference from blood components.

### 2. Introduction of the Mosquitoes Into the Secure Laboratory (Level 2+)

On day D-1, the female mosquitoes are divided into groups, placed in the engorgement boxes with moist cotton-wool on each box and introduced into the secure laboratory. The mosquitoes are kept at controlled temperature and humidity (T=25° C.±2, RH 80%±10).

### 3. Introduction of the HCV Into the L2+ Laboratory

On day D0, the viral solution containing the HCV (serum of a patient with chronic hepatitis with 2^{E}6 copies/ml) is brought by special transporter to L2+ where the mosquitoes are located. The viral solution is provided by the Molecular and Structural Virology Laboratory of the Grenoble Faculty of Medicine. The viral solution is kept in the L2+ deepfreeze at −80° C.

### 4. Preparation of the Infected Blood Solution

On day D0, the non-infected washed red cells (sheep's blood) and the viral solution is mixed in such a way as to obtain a sufficient titre to infect the mosquitoes (˜1500 copies/mosquito). ATP is added to make the blood more attractive and increase the desire of the females for it. Each mosquito takes between 3 and 5 μl per blood meal.

### 5. Infection of the Groups of Females

On day D0, the engorger containing the infected blood warmed to 37° C. is presented to the females, which have been deprived of glucose for 24 hours. The engorger is placed on each box and left in place for about 4 hours for blood meals to be taken. In parallel, the engorger with non-contaminated blood is presented to negative control females. At the end of the period of engorgement, the females are recovered using a mouth aspirator, anaesthetised with chloroform vapour and sorted under a binocular microscope. Females that have engorged themselves with blood are put back into the cages. A suitable site is left in each metal cage for the females to lay their eggs (a container of water for the Aedes females and moist earth for the Culex females). From this day the metal cages containing the engorged females are not opened or handled until the mosquitoes are sacrificed.

### 6. Maintaining the Groups of Females

The groups of females are kept at a constant temperature of about 28° C. with controlled humidity of at least 70%. Each day, the females are fed using cotton-wool impregnated with a 10% glucose solution placed on the metal cage. The laying sites are humidified through the cage using a wash-bottle.

### 7. Treatment of the Females

On the day of collection, the mosquitoes are sacrificed by putting the metal cage in the cold. The mosquitoes are then put into 1.5 ml tubes, labelled and put into the deep-freeze until the RNA extraction stage before the PCRs. The negative controls, engorged with blood containing no viruses, are also sacrificed. The groups of mosquitoes sacrificed on day D0 and put into the deepfreeze at −80° C. form the D0 positive controls of engorgement, extraction and amplification of the viral RNA.

### II—Detection of the Viral RNA

8. Extraction of the RNA from the Infected Organs

Extraction of the HCV viral RNA from mosquito tissues is carried out under a hood, using the “High Pure RNA Tissue Kit” marketed by Roche.

### 9. Amplification of the Viral RNA

The primers used, as well as the probe (for the qPCR), are designed to amplify the conserved region of the non-coding 5′ end of the viral genome. We used the sense primer 2CH (5′-AAC TAC TGT CTT CAC GCA GAA-3′) (SEQ ID. 1), located between nucleotides −289 and −269, and the antisense primer ITS (5′ GCG ACC CAA CAC TAC TCG GCT-3′) (SEQ ID. 2), located between nucleotides −70 and −90. The probe used for the qPCR was TM416 (5′-6Fam-AAC CCG CTC AAT GCC TGG A-Tamra-3′) (SEQ ID. 3) situated between nucleotides −137 and −119.

The composition of the reaction mixture for the classic RT-PCR and the qRT-PCR is summarised in the following table:

| | Classic RT-PCR | | | Real time RT-PCR | | |
|---|---|---|---|---|---|---|
| | | | Volume per | | | Volume per |
| | Initial conc. | Final conc. | tube (μl) | Initial conc. | Final conc. | tube (μl) |
| Buffer | 5 X | 1 X | 12.5 | 5 X | 1 X | 4.8 |
| dNTP | 10 mM | | 2 | 10 mM | | 0.8 |
| Sense primer | 10 μM | 0.6 μM | 0.75 | 10 μM | 0.6 μM | 0.3 |
| Antisense | 10 μM | 0.6 μM | 0.75 | 10 μM | 0.6 μM | 0.3 |
| primer | | | | | | |
| Probe | — | — | — | 25 μM | 0.312 μM | 0.05 |
| Water qs | — | — | 19.8 | — | — | 7.95 |
| final volume | | | | | | |
| RNase OUT | | | 1.2 | | | 0.4 |
| RNA master | | | 3 | | | 1.2 |
| Mix | | | | | | |
| Matrix (RNA) | — | — | 10 | — | — | 4 |

| | |
|---|---|
| Final volume: 50 μl | Final volume: 20 μl |

The RT-PCR was carried out under the following conditions: Retrotranscription occurred for 30 minutes at 50° C., followed by a stage deactivating the retrotranscriptase (15 minutes at 95° C.), then 45 PCR cycles alternating an elongation stage (1 minute at 63° C.) and a denaturation stage (30 seconds at 90° C.).

### Results

A first series of experiments revealed the presence of the viral RNA in Aedes vexans after rearing for 21 days. The results are illustrated in FIG. 1, which shows detectable quantities of the virus at day 21.

A second series of experiments was carried out in parallel on Culex pipiens (FIG. 3), Aedes vexans and Aedes caspius (FIG. 2). The results obtained in the previous experiment for Aedes were confirmed. Analysis using real time RT-PCR of the total RNA extracted from mosquitoes engorged with virus-containing blood after rearing for 24 days clearly shows the presence of viral RNA from the HCV genotype 1b, whereas it was totally absent in the negative control individuals (FIG. 2).

These experiments show that:

A. It is possible to detect the viral RNA present in the mosquitoes, which validates the extraction process of the total RNA from the complete mosquitoes and the amplification by real time RT-PCR of the viral genome (traces 14, 15 and 16, FIG. 2).

b. Viral RNA is totally absent from individuals of the Culex genus reared up to the 28^{th} day (FIG. 3).

c. Finally, it is out of the question that the RNA detected is residual RNA, due to the total absence of this RNA in the Culex mosquitoes up to D28 (FIG. 3, traces 6 to 17), just as it being the result of contamination can also be excluded in view of the absence of any detectable viral RNA in the negative controls.

### BIBLIOGRAPHY

(1) Bartenschlager R, Frese M, Pietschmann T (2004). Novel insights into hepatitis C virus replication and persistence. Advances in Virus Research. 63: 71-180.

(2) Bartenschlager R, Lohmann V (2001). Novel cell culture systems for the hepatitis C virus. Antiviral Research. 52(1):1-17.

(3) Bartosch B, Dubuisson J, Cosset F L (2003). Infectious hepatitis C virus pseudo particles containing functional E1-E2 envelope protein complexes. Journal of Experimental Medicine. 197(5): 633-42.

(4) Baumert T F, Ito S, Wong D T, Liang T J (1998). Hepatitis C virus structural proteins assemble into viruslike particles in insect cells. Journal of Virology. 72(5): 3827-36.

(5) Chang T T, Chang T Y, Chen C C, Young K C, Roan J N, Lee Y C, Cheng P N, Wu H L (2001). Existence of hepatitis C virus in Culex quinquefasciatus after ingestion of infected blood: experimental approach to evaluating transmission by mosquitoes. Journal of Clinical Microbiology. 39 (9): 3353-5.

(6) Flint M, Quinn E R, Levy S (2001). In search of hepatitis C virus receptor(s). Clinical Liver Diseases. 5(4): 873-93.

(7) Fouque F., Vazeille M, Mousson L, Gaborit P, Carinci R, Issaly J, Rodhain F, Failloux A B (2001). Aedes aegypti susceptibility to a Dengue virus. Tropical Medicine and International Health 6 (1): 76-82.

(8) Germi R, Crance J M, Garin D, Zarski J P, Drouet E (2001). Hepatitis C virus culture systems. Pathologie-Biologie. (Paris). 49(3) : 255-61.

(9) Germi R, Crance J M, Garin D, Guimet J, Thelu M A, Jouan A, Zarski J P, Drouet E (2001). Mosquito cells bind and replicate hepatitis C virus. Journal of Medical Virology. 64 (1): 6-12.

(10) Silverman A L, McCray D G, Gordon S C, Morgan W T, Walker E D (1996). Experimental evidence against replication or dissemination of hepatitis C virus in mosquitoes (Diptera:Culicidae) using detection by reverse transcriptase polymerase chain reaction. Journal of Medical Entomology. 33 (3): 398-401.

(11) Wakita T et al. (2005). Production of infectious hepatitis C virus in tissue culture from a cloned viral genome. Nature Medicine. 11(7): 791-6.

1. A method for amplifying the hepatitis C virus (HCV) in vivo, comprising:
ingesting of the viral source by female Aedes vexans or Aedes caspius mosquitoes; and
rearing the mosquitoes for an amount of time necessary to amplify the virus.

2. The method according to claim 1, wherein the mosquitoes are obtained from wild strains.

3. The method according to claim 1, wherein the viral source is supplemented with red blood cells, and optionally ATP.

4. The method according to claim 1, wherein ingesting comprises ingesting a blood meal comprising the viral source.

5. The method according to claim 16, wherein the engorger is covered by chicken skin.

6. The method, according to claim 1, wherein the viral source is serum.

7. The method according to claim 1, wherein the viral source consists of attenuated virus.

8. The method according to claim 1, wherein the ingesting is at a level of at least 1000 copies of the virus/mosquito.

9. The method according to claim 1, wherein rearing comprises rearing the mosquitoes for a period of between 15 and 30 days.

10. The method according to claim 1, further comprising after rearing recovering the virus from mosquito tissue.

11. Tissue from Aedes carrying HCV, obtained by the process according to claim 1.

12. An isolated Aedes cell carrying HCV, obtained by the process according to claim 1.

13. A method for obtaining cells producing HCV in vitro comprising culturing cells according to claim 12 in the presence of cells of type AP61 or C6/36.

14. An isolated mosquito cell producing HCV in vitro, obtained by the process according to claim 13.

15. (canceled)

16. The method of claim 4, wherein ingesting a blood meal comprises providing an engorger comprising the blood meal.

17. The method of claim 6, wherein the serum is of human origin.

18. The method of claim 9, wherein rearing comprises rearing for about 20 days.
